(19) United States
(12) Patent Application Publication
(10) Pub. No.: US 2010/0331336 A1
(43) Pub. Date: Dec. 30, 2010

(54) PROTECTIVE EFFECT OF HIGH DOSE FOLATE ON MYCARDIAL ISCHEMIA

(75) Inventor: An Moens, Brasschaat (BE)

Correspondence Address: TRASKBRITT, P.C., P.O. BOX 2550, SALT LAKE CITY, UT, 84110, US

(73) Assignees: Universiteit Antwerpen, Antwerperpen (BE); BioMedbooster, Maastricht (NL)

(21) Appl. No.: 12/735,550

(22) PCT Filed: Feb. 6, 2009
(86) PCT No.: PCT/EP2009/051357

§ 371 Date: Aug. 31, 2010

Related U.S. Application Data
(60) Provisional application No. 61/063,750, filed on Feb. 6, 2008.

(51) Int. Cl. A61K 31/519; A61P 9/10; A61P 9/06; C07D 475/04

(52) U.S. Cl. 514/249; 544/261

(57) ABSTRACT

The present invention relates to the use of high dose of folic acid, or an equivalent dose one of its biological active derivatives to blunt myocardial dysfunction during ischemia and to ameliorate post-reperfusion injury. The invention specially relates to an early treatment by the application of a high dose of at least 200 mg folic acid, or an equivalent dose of a derivative during active ischemia, before the reperfusion.

The present invention relates to the use of high dose of folic acid, or an equivalent dose one of its biological active derivatives to blunt myocardial dysfunction during ischemia and to ameliorate post-reperfusion injury. The invention specially relates to an early treatment by the application of a high dose of at least 200 mg folic acid, or an equivalent dose of a derivative during active ischemia, before the reperfusion

During acute coronary occlusion, myocardial perfusion and oxygen delivery become insufficient to support active muscle contraction. In the ischemic zone, myocardial high energy phosphate content falls, inorganic phosphate rises, and the tissue is rendered acidotic (Jennings and Steenbergen, 1985), resulting in regional dyskinesis and global dysfunction. Coronary reperfusion after ischemia limits tissue damage but confers toxicity from activation of reactive oxygen species (ROS), calcium-dependent proteases such as calpain, myofilament contracture, microvascular dysfunction, and inflammatory cytokines (Gross and Gross, 2006). ATP depletion during ischemia may also contribute to reperfusion injury (Gunduz. et al., 2006) In addition, ROS generated during both ischemia (Klawitter et al., 2002) and reperfusion (Zweier and Talukder, 2006) is thought to play a central role, and various anti-oxidant strategies have been tested to offset this damage.

One approach to limiting ischemia/reperfusion damage is to subject hearts to brief ischemia prior to more prolonged exposure, a phenomenon termed ischemic pre-conditioning. This involves multiple mechanisms including protein kinase C activation (Yamamura et al., 2005), stimulation of mitochondrial K_{ATP} channels, and enhanced nitric oxide synthesis (Jones and Bolli, 2006), with the latter playing a central role. Infarct size after ischemia-reperfusion is greater in eNOS deficient mice (Jones et al., 1999) but reduced in mice over-expressing eNOS (Jones et al., 2004) Reduced NO synthesis and increased ROS generation by NOS occur when it becomes functional uncoupled by depletion/oxidation of its obligate cofactor tetrahydrobiopterin (BH4) (Hevel and Marietta, 1992; Vasquez-Vivar et al., 1998)

The potential role of NOS in IR injury has led to efforts to enhance enzyme function, including administration of BH4 in vitro (Wajima et al., 2006). A far less costly alternative maybe folic acid (FA), a B-vitamin important for normal mitochondrial protein and nucleic acid synthesis (Depeint et al., 2006) but that also stabilizes BH₄ by augmenting its binding-affinity to eNOS (Stroes et al., 2006; Hyndman et al., 2002) and enhances BH₄ regeneration from oxidized and inactive BH₂. FA or its active metabolite—5-methyltetrahydrofolate (5-MTHF)—improves endothelial function Verhaar et al., 1998; Shirodaria et al., 2007; Moat et al., 2006).

Low doses of FA have been tested in patients with diseases of the cardiovascular system, but recent clinical studies testing the utility of FA for chronic cardiovascular risk reduction have been somewhat disappointing (Bazzano et al., 2006) and are certainly not conclusive. Indeed, FA has been studied in clinical trials, particularly to test its potential to lower cardiovascular risk in patients with myocardial vascular disease. For example, Oster (1981) demonstrated that long-term folic acid treatment (˜10 years) at a dose far higher than typically used (40-80 mg/day), reduced the incidence of myocardial infarction, angina pectoris and requirement for nitroglycerin in patients with coronary artery disease. This observation was not confirmed by a placebo controlled trial nor was the mechanism explored. Other studies focused on the ability of FA to lower homocysteine, and found a 3 μmol/l decrease in serum homocysteine (achievable with 0.8 mg/d). Despite these positive results, meta-analyses of multiple FA trials for use in cardiovascular prevention have been unimpressive (JAMA 2002; Wang et al., 2007; Wald et al., 2006) and it remains unclear if dose, study duration, target population, or other factors explain this. The doses used in prior cardiovascular interventional studies (5-25 mg/70 kg/d) or prevention trials (500 μg-1 mg) are low, compared with the dose of the invention.

WO0130352 describes a dose of 30-500 mg folate, preferably 30-100 mg folate to treat hyperhomocystemia. Although hyperhomocystemia is considered as a risk factor for cardiovascular diseases, the mechanism how high levels of homocysteine would lead to cardiovascular diseases is unknown, and the application does not describe any effect on cardiovascular diseases as such, especially not when the patient is not suffering from hyperhomocystemia.

WO2006113389 describes a method for improving vascular dilation comprising administering to the subject a high dose (20-100 mg) of folic acid, and claims that a daily dose of 20-100 mg may delay or minimize development of a single heart disease. There is no indication that the myocardial dysfunction during ischemia may be blunted, not that the post-reperfusion injury may be ameliorated by this treatment.

Surprisingly we found that high dose (at least 200 mg per subject) FA pre-treatment and/or treatment during ischemia before reperfusion can ameliorate IR injury and explored the mechanisms for such an effect. The data show post-reperfusion benefits but more strikingly reveal a marked and surprising effect of FA-pre-treatment on reducing regional and chamber dysfunction and ROS generation during the period of ischemia itself. This benefit appears linked to alterations in purine catabolism and preservation of high energy phosphate levels during ischemia.

In this study we demonstrated for the first time that pretreatment with high doses of oral folic acid markedly reduced the severity of ischemic dysfunction during coronary occlusion, and enhanced function and diminished infarct size following reperfusion. These effects were linked to preserved high energy phosphates during ischemia despite flow reduction, improving global and regional function, reducing necrosis and oxidative stress, and preserving eNOS coupling upon reperfusion. The ability of FA pre-treatment to help sustain myocardial contraction despite substantially reduced coronary flow is unusual, and quite different from the influence of anti-oxidants and classical preconditioning agents. These typically have little impact during ischemia, but benefit the heart post-reperfusion. For that matter, the benefit of FA on reducing infarct size even if delivered after ischemia had commenced implies a different mechanism.

A first aspect of the invention is the use of a high dose folic acid of at least 200 mg, preferably at least 600 mg, even more preferably at least 1000 mg or an equivalent dose of a folic acid derivative to blunt myocardial dysfunction during ischemia and/or to ameliorate post-reperfusion injury. Folic acid derivatives as used here are known to the person skilled in the art and include, but are not limited to folate and 5-methyltetrahydrofolate. Myocardial dysfunction as used here includes, but is not limited to decreased myocardial contractility, myocardial cell death and/or infarct induced arrhythmias. One preferred embodiment is the use of a high dose folic acid or an equivalent dose of a folic acid derivative whereby said myocardial dysfunction is decreased myocardial contractility. another preferred embodiment is the use of a high dose folic acid or an equivalent dose of a folic acid derivative whereby said myocardial dysfunction is myocardial cell death. Still another preferred embodiment is the use of a high dose folic acid or an equivalent dose of a folic acid derivative whereby said myocardial dysfunction is infarct induced arrhythmias. Preferably the high dose is administered as a single dose. Even more preferably, the dose is administered during the ischemia, before reperfusion. In one preferred embodiment, the administration is oral administration. In another preferred embodiment, the dose is administered by intravenous injection. Alternatively, the dose may be administered transdermally, or by intramuscular injection.

Another aspect of the invention is the use of a high dose folic acid of at least 200 mg, preferably at least 600 mg, even more preferably at least 1000 mg, or an equivalent dose of a folic acid derivative as an early treatment during active ischemia before reperfusion. Early treatment as used here means that the treatment is started after the start of the active ischemia, but before the reperfusion. Preferably the high dose is administered as a single dose. Even more preferably, the dose is administered during the ischemia, before reperfusion. In one preferred embodiment, the administration is oral administration. In another preferred embodiment, the dose is administered by intravenous injection. Alternatively, the dose may be administered transdermally, or by intramuscular injection.

Still another aspect of the invention is a pharmaceutical composition, comprising a single dose of at least 600 mg folic acid, preferably a single dose of at least 1000 mg folic acid, or an equivalent dose of a folic acid derivative, possibly in combination with a pharmaceutical acceptable vector. A pharmaceutical composition, as used here, can be a liquid composition suitable for injection, or a solid composition for oral intake.

Still another aspect of the invention is the use of folic acid or an equivalent dose of a folic acid derivative as a cardioprotective or therapeutic agent to improve or restore decreased high energy phosphate levels in cardiovascular disorders with decreased ATP/ADP-levels. Preferably said use of folic acid is the use of a high dose. Even more preferably, said high dose is at least 200 mg, most preferably at least 600 mg. Improve as used here means that the high energy phosphate level is increased by the treatment, be it not to the normal level in a healthy person. Restore, as used here, means that the high energy phosphate level after treatment is comparable to that of a healthy person, or higher.

### BRIEF DESCRIPTION OF THE FIGURES

FIG. 1: Cardiac function measured by pressure/volume-loop analysis. A] Example of pressure-volume loops at baseline (1), end of ischemia (2), and 90 min reperfusion (3) for a placebo (upper) and FA pre-treated (lower) animal. FA pre-treatment improved function both during ischemia and following reperfusion. B] Summary hemodynamics for the full IR protocol shows the time course for systolic and diastolic parameters. (p values are from repeated measures ANOVA, time×treatment interaction/treatment effect).

FIG. 2: Open chest echocardiography. The upper left panel shows examples of M-mode echocardiography at baseline and after 30 min of ischemia in the placebo and FA-treated group. Summary plots show results obtained from these echocardiograms versus time. Ejection fraction and anterior-septal wall-thickening declined in the placebo group but were unchanged in the FA-group (p-values are for treatment effect by RMANOVA).

FIG. 3: HPLC-analysis of parameters of high energy phosphate (HEP) metabolism and redox-state. Under baseline conditions, FA pre-treatment did not alter HEP (ATP, ADP, AMP), but did increase inositol monophosphate (IMP) and its catabolites (oxypurines: xanthine, hypoxanthine and uric acid). After 30 minutes of ischemia, the levels of HEP were better maintained in the FA-treated hearts compared with placebo animals. While oxypurines rose markedly after ischemia in placebo-treated animals, this was not observed in the FA-treated rats. P-values are from a 2-way ANOVA, with the first value indicating the effect of ischemia, and the second the interaction between treatment group and ischemia.

FIG. 4: Effect of folic acid on myocardial necrosis A] Oral (7 days) FA pre-treatment reduced infarct size in vivo (*: p<0.001; Mann-Whitney). Upper panel: Area at risk (AAR) was comparable between all groups. Middle panel: Infarct size, expressed as % AAR was significantly reduced in all FA-groups. Lower panel: plot-graph of the individual correlation between myocardial necrosis and AAR. B] FA pre-treatment in vivo reduces contraction band necrosis (*p=0.001). C] FA pretreatment in vivo reduces apoptosis (TUNEL-staining) (*p=0.005 and p=0.001). D] FA pre-treatment reduced myocardial necrosis by ˜80% in hearts studied in vitro. (*: p<0.0001). E] Increased lactate dehydrogenase (LDH) after ischemia-reperfusion peaked at 10-20′, whereas hearts receiving FA pre-treatment had minimal LDH release. (p<0.001).

FIG. 5: Effects of FA treatment on ROS generation. A] Lucigenin-enhanced superoxide detection shows marked reduction of ischemia and IR-induced superoxide with FA pre-treatment. B] O₂⁻ formation in control-vehicle treated IR hearts was markedly suppressed by acute addition of BH₄, whereas this was blunted by nearly half in myocardial extracts obtained from FA-pre-treated hearts. C-D] DHE and E] DCF stained myocardium reveal increased ROS generation in ischemic or IR myocardium that was substantially reduced in heart receiving FA pre-treatment. F] Direct antioxidant effects of FA compared with Tempol. Superoxide was generated by xanthine/xanthine oxidase in vitro, and measured by lucigenin-enhanced chemiluminescence at varying added concentrations of either FA or tempol.

FIG. 6: Effect of FA pre-treatment on NO-pathway A] SDS-Page gel shows increased eNOS monomer in IR myocardium that was reduced towards control levels by FA pre-treatment. B-C] Summary densitometry data of gel analysis as shown in panel A. FA pre-treatment reduced the monomer/dimmer ratio, but had no effect on total eNOS protein expression. D] Bradykinin administered to hearts in vitro before versus after IR showed a marked decline in endothelium-dependent flow response. This was restored to a normal response by pre-treating with FA. E] FA treatment did not influence the flow-response to sodium nitroprusside, supporting that the disparity observed with bradykinin was endothelium-dependent.

### EXAMPLES

### Materials and Methods to the Examples

### Ethical Committee

Experiments were performed in accordance with the Guide for the Care and Use of Laboratory Animals published by the US National Institute of Health (NIH-Publication, N° 85-23) and approved by the Ethical Committees of the University of Antwerp and by the Johns Hopkins Medical Institutions.

In vivo Ischemia Model

Adult Wistar rats received FA (10 mg/day unless otherwise stated) or placebo by oral gavage for 7-days prior to performing the ischemia/reperfusion (IR) experiment. A total of 131 rats were used, the sample size reflecting the requirement for multiple assays that could not be performed in each animal, but were rather obtained in different subsets of the total group. Animals were anesthetized (pentobarbital 60 mg/kg), intubated via tracheotomy, and ventilated (Harvard Apparatus, Mass.). ECG was monitored and temperature maintained at 37.5° C. The left anterior artery was exposed through the 4-5^{th} intercostal space and a suture placed around it, and transient coronary artery ligation performed for 30 min with (n=85) or without (n=46) 90 min reperfusion. In one subgroup of reperfused animals (n=9), FA was provided 10 minutes after the onset of LAD occlusion (i.e. 20 minutes before the onset of reperfusion), delivered as an IV-bolus.

### In Vivo Hemodynamics

LV function was assessed in vivo by pressure-volume loops (n=14) during both ischemia and following reperfusion. A 1.4 F pressure-volume catheter (SciSense, London-Ontario, Canada) was advanced through the apex, positioned along the longitudinal axis, and attached to a stimulator/analyzer (IOX 1.8.9.19, Emka Tech., Paris). Volume data were calibrated using the hypertonic saline method, assuming a gain=1. Two animals had catheter dislocation during ischemia, and their volume data were not used. Open chest myocardial anterior wall motion was also measured using a Sequoia—Acuson C256 equipped with 15 MHz linear transducer (Sequoia C256 Echocardiography System, Acuson Corporation, Mountain View, Calif.) at the parasternal view of the left ventricle chamber as described.

### Assessment of Redox and Energy Metabolism

Snap frozen samples (n=24) from the anterior wall (±FA pre-treatment; ±30 min ischemia, no reperfusion) were de-proteinized and subjected to HPLC analysis of water-soluble low-molecular weight compounds reflecting tissue oxid-oreductive and energy status. High-energy phosphates, oxypurines (hypoxanthine, xanthine, and uric acid), nucleosides (inosine and adenosine), malondialdehyde, and reduced and oxidized glutathione were measured by ion-pairing HPLC as described (Takimoto et al., 2005).

### Myocardial Flow Measurements

Regional myocardial blood flow was assessed (n=12) by nuclear-activated microspheres (15 μM diameter, BioPal, Worcester, MA) injected in the left atrium (0.3 ml of 2.5*10⁶ spheres/ml) at baseline and after 5 and 30 min of ischemia (Reinhardt et al., 2001). Total counts per minute (cpm) were normalized to weight and results from the ischemic zone normalized to the remote region to provide relative blood flow before and during ischemia.

### In Vitro IR Model

Adult Wistar rats (n=28) were anesthetized (pentobarbital 60 mg/kg), and hearts rapidly excised and mounted onto a retrograde perfusion system (Emka, Paris, France) using warmed, oxygenated buffered Krebs-Henseleit solution at constant perfusion pressure (75 mmHg). Hearts were paced at 300 bpm, and maintained in an unloaded state. Coronary flow was measured by an in-line ultrasonic flow probe (Transonic Systems, Ithaca, N.Y., USA). After 30 min equilibration, vasodilators drugs (bradykinin and sodium nitroprusside) were infused by bolus injection (50 μl, 10⁻⁸-10^{-5.5} M i.c.) and coronary flow reserve assessed at constant perfusion pressure. After re-establishing baseline, hearts received FA (4.5 10⁻⁶M i.c.) or vehicle for 30 min. Afterwards the hearts were subjected to 40-min zero coronary flow followed by 40 min of reperfusion. Coronary effluent was collected, concentrated (Sartorius-Sipan, Lier, Belgium) and analysed for lactate dehydrogenase (Vitros 950AT, O. C. D., Beerse, Belgium). Coronary vasodilator responses to the same two agents were repeated following reperfusion.

### Infarct Size Analysis and Histology

Infarct size was assessed in 31 rats, with area at risk (AAR) determined by negative staining with Evans blue injected during coronary occlusion, and subsequent staining with TTC to detect myocardial necrosis. LV (free wall+whole septum), AAR (neg Evan's Blue) and area of necrosis (TTC-white) were measured by planimetry (Soft Imaging system GmbH, Analysis pro version 3.00), and digitized images subjected to equivalent background subtraction, brightness, and contrast enhancement for improved clarity and sharpness. For in vitro studies, TTC staining was not preceded by Evans' blue staining because global ischemia was created. Contraction band necrosis was assessed in vivo rat hearts (n=22) fixed in Carnoy solution and stained with Masson's trichrome. Serial adjacent fields were examined throughout the full LV to calculate the percent myocardium with contraction bands present. Similar analysis was performed for TUNEL-positivity (Chemicon Intern, Temecula, Calif.), also expressed as percent LV area.

### ROS Determination

Superoxide was assessed by lucigenin (5 μM)-enhanced chemiluminescence (Beckman LS60001C, n=23)²² and fluorescent microtopography (dichlorodihydro-fluorescein diacetate, DCF, n=16) and dihydroethidium, DHE, n=24) (Gupte et al., 2005). The direct anti-oxidants effects of folic acid were analysed using an in vitro xanthine/xanthine oxidase system (Antoniades et al., 2006) and compared with Tempol.

eNOS Monomer/Dimer Formation and Enzymatic Activity

SDS-resistant eNOS dimers and monomers were assayed on ischemia/reperfusion-tissue (n=16) using low temperature SDS-PAGE as previously described (Takimoto. et al., 2005) NOS enzymatic activity was assessed by arginine to citrulline conversion assay from extracts obtained from frozen myocardium (n=15) (Takimoto. et al., 2005).

### Data Analysis

Data are presented as mean±sem with p-values<0.05 considered statistically significant. Coronary flow-reserve and in vivo hemodynamic data were analyzed by repeated measures ANOVA. Other comparisons used either 1-way ANOVA or Kruskal-Wallis tests to compare between multiple independent groups, with a Bonferroni correction for multiple comparisons. Analysis utilized SPS6 version 11.0 (Chicago, Ill.).

### Example 1

### Folic Acid Pre-Treatment Improves Cardiac Function with I/R

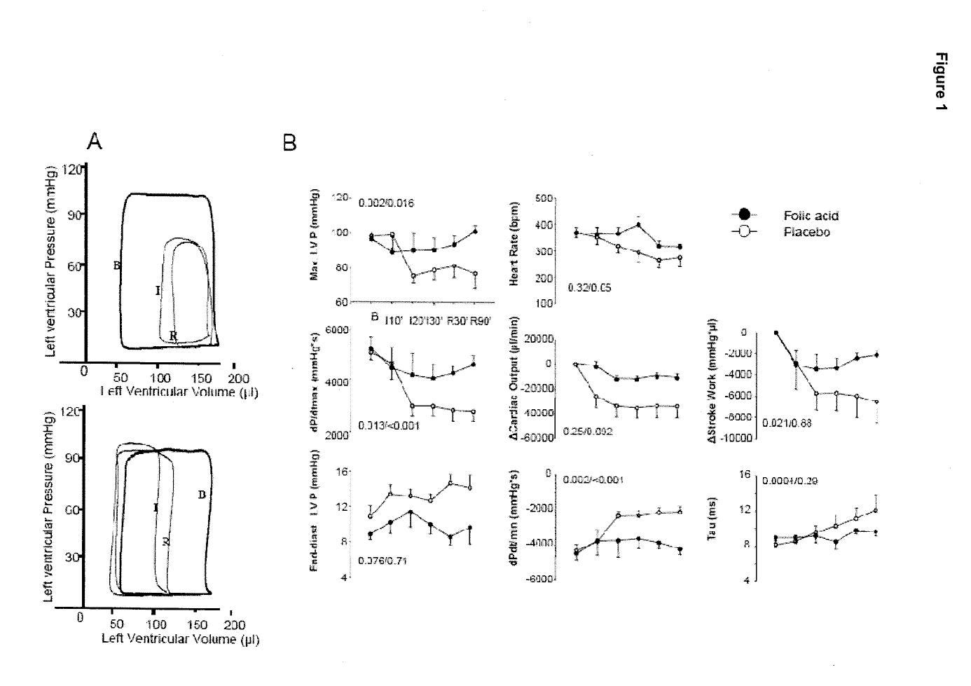

FIG. 1A displays example PV relations and summary data for systolic and diastolic ventricular function in hearts with or without FA (10 mg/d) pre-treatment. Data were measured at initial baseline in open-chest rats, during 30 minutes of coronary occlusion, and after 90 minutes of reperfusion. Control hearts displayed markedly reduced cardiac function with a rightward downward shift of the PV loops after 30 min of LAD occlusion that persisted after reperfusion (upper panel). With FA pre-treatment, systolic and diastolic function was better preserved during ischemic and reperfusion periods (lower panel). Summary data (FIG. 1B) supports these examples. Peak LV pressure (i.e. systolic blood pressure) changed little despite LAD occlusion in FA pre-treated rats but fell by nearly 25% in controls. Similar disparities were observed in dP/dtₘₐₓ. Cardiac output and stroke work also declined less in FA-pretreated animals, particularly in late ischemia and reperfusion. Thus, cardiac function was enhanced during the ischemic period and after reperfusion by FA pre-treatment.

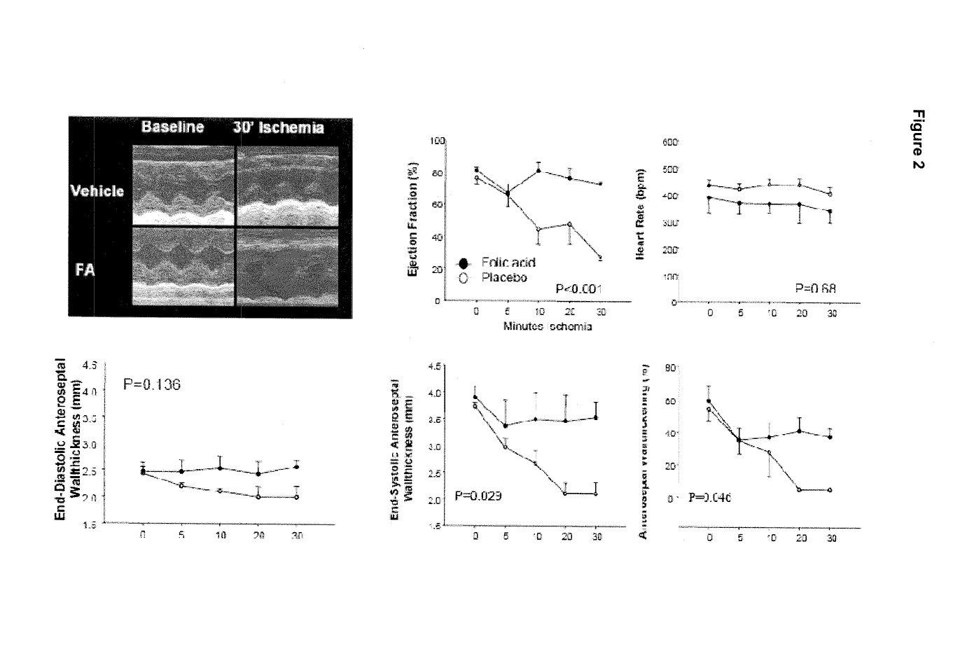

The relative preservation of global function during ischemia was somewhat surprising, and suggested regional dysfunction despite coronary occlusion in FA pre-treated hearts. To test this, we performed open-chest echocardiography to measure wall thickening and time course of dysfunction during the 30 minutes of LAD occlusion (FIG. 2). Example M-mode tracings (upper left panel) shows marked reduction of anterior wall thickening during ischemia in controls but preserved thickening in FA treated animals. Ejection fraction was much higher despite ischemia (72.8±1.2% vs. placebo: 27.4±2.2%, p<0.001 at 30 min), consistent with the PV-loop results, as was anterior-septal thickening (37.±5.3% vs. placebo 5.1±0.6%, p=0.004). Thus, FA pre-treatment enhanced regional dysfunction in the ischemic zone despite coronary occlusion, and this persisted or was further improved after reperfusion.

### Example 2

### Folic Acid and Myocardial Flow

Since FA pre-treatment improved both regional and global function during LAD occlusion, we tested whether it enhanced myocardial blood flow to reduce the ischemic insult per se. However, after 5 min of LAD ligation, the ratio of ischemic/remote zone myocardial perfusion obtained by microsphere analysis declined similarly in both placebo and FA-pre-treated groups (−73.7±6.0% and −77.7±5.1%, respectively). Flow remained low in both groups at 30 minutes (−78.4±9.3% vs. placebo −71.2±13.8% compared to the remote region).

### Example 3

### Folic Acid Preserves Myocardial Levels of High Energy Phosphates

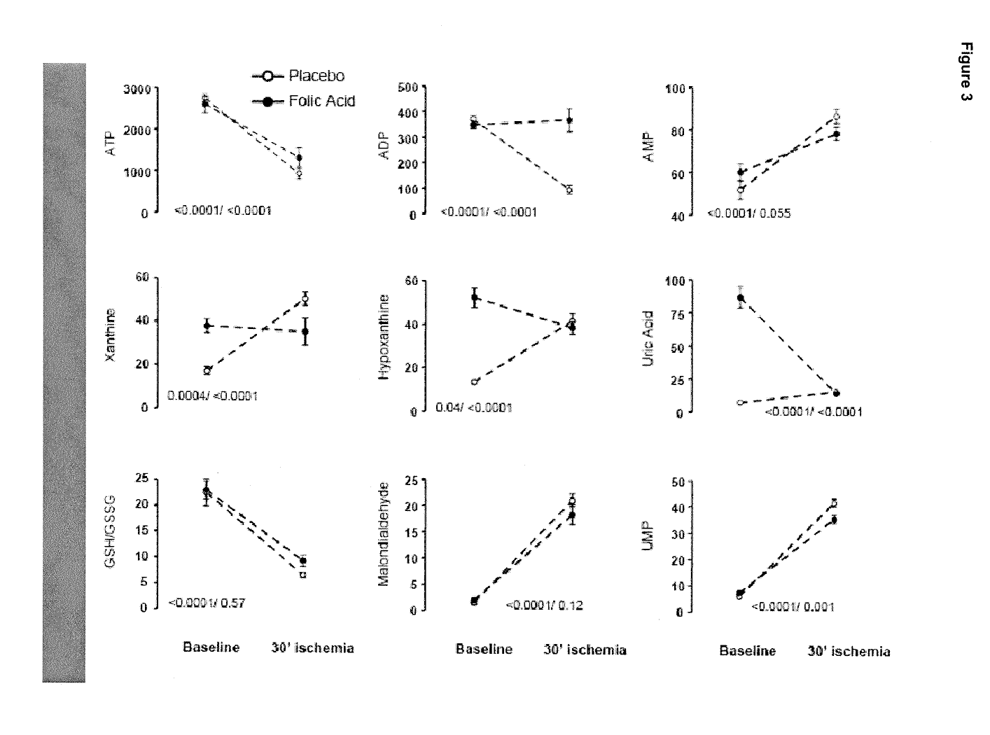

Since improved perfusion could not explain the FA-treatment effect, we next tested whether FA altered high energy phosphate (HEP) metabolism at baseline and particular during ischemia. As shown in FIG. 3, FA-pretreatment did not alter HEP at baseline, but did elevate levels of inositol monophosphate (IMP) and its catabolites (oxypurines: xanthine, hypoxanthine, uric acid). During ischemia, myocardial ATP and ADP declined more than 66% in controls, consistent with reported changes¹. However, both were maintained at higher levels in FA pre-treated hearts, (p<0.001 for interaction of FA treatment and ischemic response). Oxypurines rose markedly during ischemia in controls, consistent with reduced HEP and enhanced AMP catabolism; however, they changed little or declined in FA-treated hearts. Lastly, we examined redox-state as indexed by malondialdehyde (a marker of lipid peroxidation) and relative reduced/oxidized glutathione ratio. Both were little changed by FA pre-treatment with or without myocardial ischemia.

### Example 4

### Folic Acid Pre-Treatment Reduces Myocardial Infarct Size

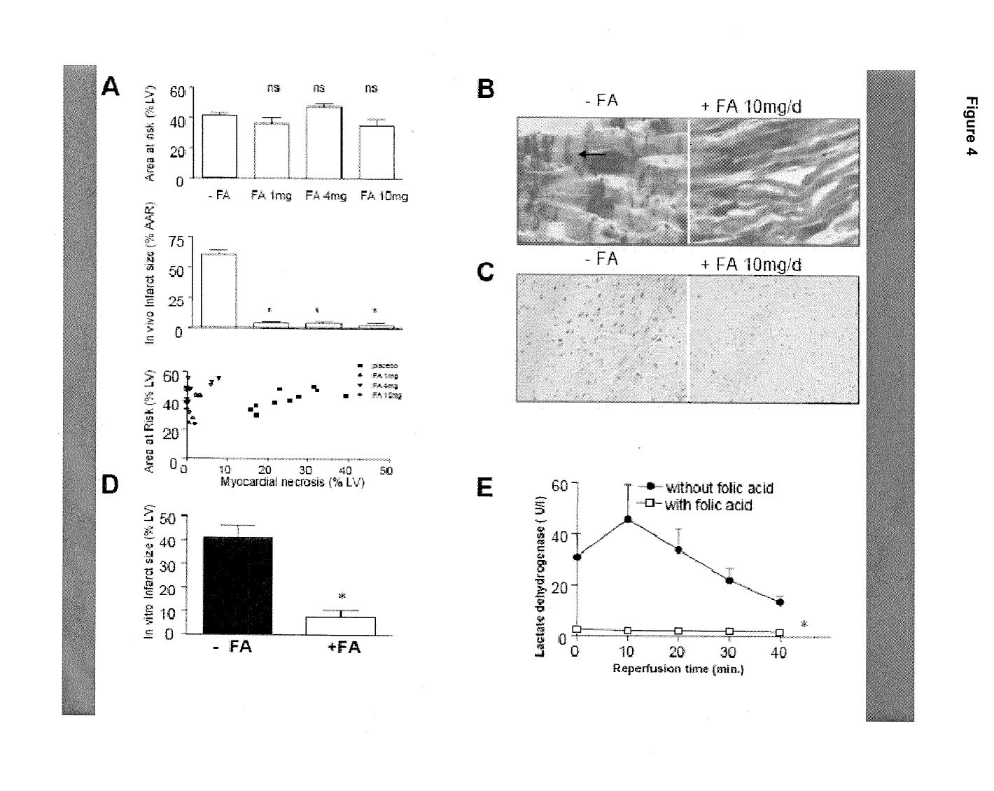

A potential consequence of improving both function and HEP metabolism during ischemia is that the subsequent infarct size is reduced. As shown in FIG. 4A, infarct size was 60.3±4.1% the area of risk in placebo-treated animals versus 3.8±1.2% with FA-pre-treatment (p<0.002). In separate studies, we found this decline in infarct size occurred with pre-treatment by 40% and even 10% of the principal FA dose (i.e. 1 and 4 mg/d). Reperfusion contraction band necrosis was observed throughout 26.7±2.6% of the LV in vehicle treated hearts, but 4.6%±1.2% in FA treated hearts (p=0.001, FIG. 4B). Similarly, TUNEL-positive myocytes were prevalent (63.0±5.8% of LV fields) with vehicle treatment, but rare (4.3±1.3%) with FA treatment (p=0.001, FIG. 4C). Ischemia triggered lethal ventricular arrhythmia in 36.7% of control rats (vs 8.3% FA-treated), while reperfusion arrhythmia occurred in 6.1% of controls and no FA treated animal (p<0.01 for both). Since post-reperfusion infarct size in vivo is partially related to coupling of function with coronary perfusion, we also tested the impact of FA-pretreatment in isolated hearts. FA reduced infarct size markedly (7.7±2.8% vs. 41.1±4.9%, respectively, p<0.0001, FIG. 4D), and there was less lactate dehydrogenase in the coronary effluent (FIG. 4E), consistent with reduced cell necrosis.

### Example 5

### Folate Pre-Treatment Vs. Acute Folate Administration

In a separate set of 9 animals, FA was administered after 10 of coronary occlusion (10 mg i.v.), the time at which functional responses appeared to first diverge (c.f. FIGS. 1, 2). Interestingly, similar benefits on reducing infarct size relative to AAR (n=5; 3.0±2.2%, p<0.001 vs. placebo) were observed, while AAR was itself similar to placebo (52.4±5.5%). Histology was performed on 4 of the animals and showed reduced TUNEL staining (4.3±1.3% LV, p<0.0001 vs. placebo) and contraction band necrosis (5.1±0.7% LV, p<0.001 vs. placebo). Thus, the FA effect on infarct reduction did not appear to be a classic pre-conditioning effect, as it could be generated by FA administration after ischemia had commenced.

### Example 6

### Folate Pre-Treatment Reduces ROS Generation

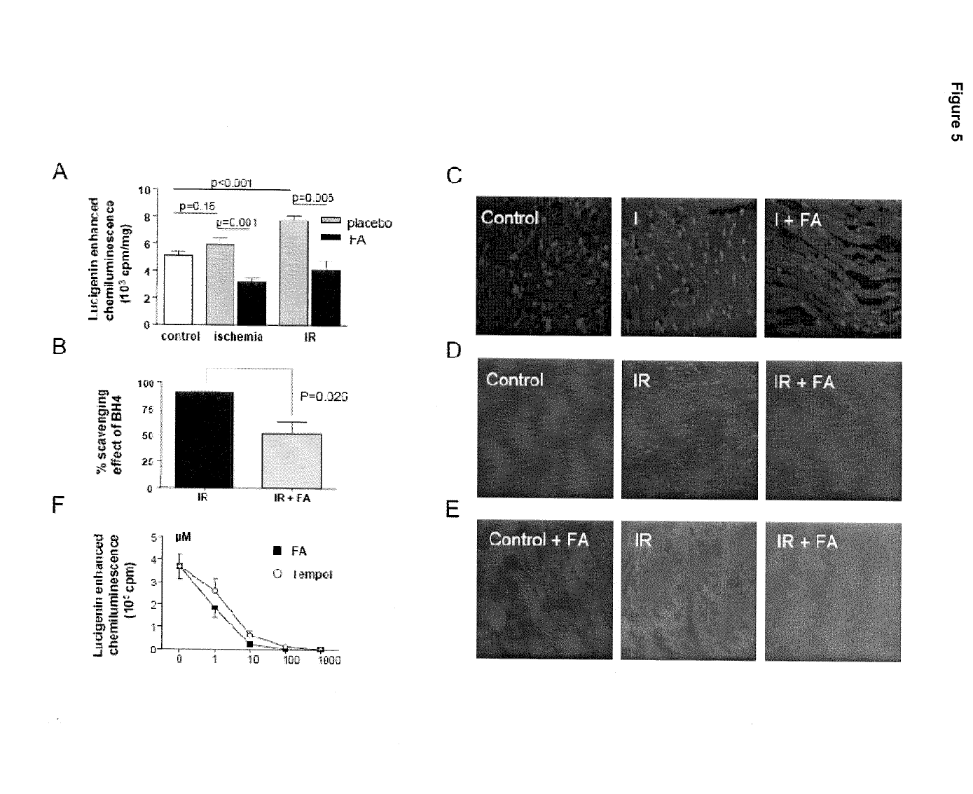

Myocardial superoxide (lucigenin chemiluminescence) declined nearly 50% in FA pre-treated animals during ischemia and after 90 minutes reperfusion (FIG. 5A). When extracts were pre-incubated with 100 μM BH4, O₂⁻ generation declined by 90.9±0.7% in vehicle treated hearts, but less so in FA pre-treated hearts (52.1±11.3%, p<0.03, FIG. 5B). This suggested that an anti-oxidant pathway targeted by BH4 (e.g. NOS coupling) was either lacking in FA-pre-treated hearts or already ameliorated by FA therapy. The effect of FA on ischemia and IR-induced ROS was further examined by oxidative fluorescent microtopography. Both DHE and DCF-stained myocardial slices showed marked ROS generation in the placebo group that was reduced with FA pre-treatment. FIG. 5C-E shows example images, mean data were obtained in n=4 for each group and consistent with these examples. To test for potential direct anti-oxidant effects of FA, we performed an in vitro assay using a xanthine/xanthine oxidase O₂⁻ generating system (FIG. 5F). FA had substantial anti-oxidant effects in this assay, similar to that from the superoxide dismutase mimetic Tempol.

### Example 7

### Folate Pre-Treatment Improves eNOS Dimerization and Activity, and Endothelial Function

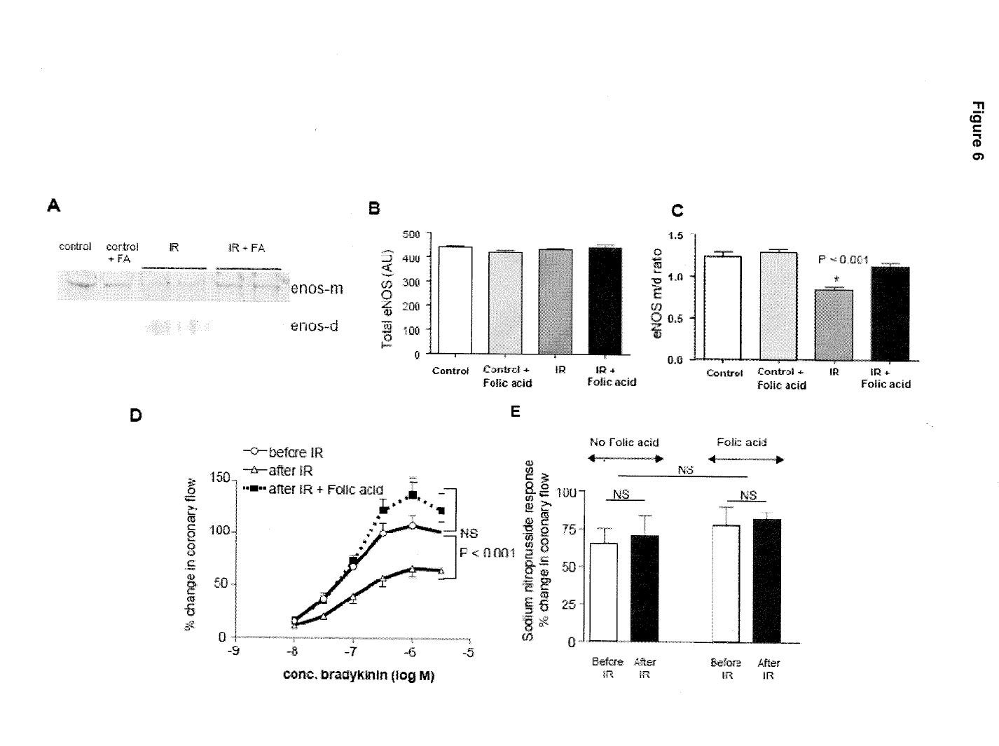

Given that FA and its active metabolite 5-MTHF are mechanistically linked to BH4-mediated improvement in NOS function, including reduction in NOS-derived superoxide, we examined whether FA influenced NOS coupling in I/R hearts. FIG. 6A shows example immunoblots for eNOS with monomer (140 kDa) and dimer (280 kDa) bands. Total eNOS (sum of both) was similar among the different conditions (FIG. 6B), however the ratio of dimer/monomer declined in vehicle treated IR hearts reflecting NOS uncoupling (p<0.001, FIG. 6C), yet was near normal in FA-pre-treated hearts. NOS activity, measured by arginine-citrulline conversion was, however borderline, not significant improved in the treated vs. non-treated hearts (p=0.08, not shown).

Coronary endothelial function also improved by FA pre-treatment. Bradykinin induced a maximal 108.3±9.2% rise in coronary flow in isolated control hearts, but only 67.1±8.1% after IR, (p<0.001 FIG. 6D). This was restored to control levels in hearts pre-treated with FA (122.0±11.3%), without any change in basal dilation. There were no changes in the coronary flow at baseline (placebo: 10.2±0.7 ml/min, FA: 11.4±13.8 ml/min, p=0.5) or after ischemia (placebo: 9.8±0.7 ml/min and FA: 8.9±1.4 ml/min, p=0.5) between groups. In contrast to bradykinin-stimulation, coronary flow increased similarly in control and ischemic conditions with sodium nitroprusside (FIG. 6E), supporting endothelium dependence of the prior effect.

### REFERENCES

Antoniades C, Shirodaria C, Warrick N, Cai S, de Bono J, Lee J, Leeson P, Neubauer S, Ratnatunga C, Pillai R, Refsum H, Channon K M. 5-methyltetrahydrofolate rapidly improves endothelial function and decreases superoxide production in human vessels: effects on vascular tetrahydrobiopterin availability and endothelial nitric oxide synthase coupling. Circulation. 2006; 114:1193-1201.
Bazzano L A, Reynolds K, Holder K N, He J. Effect of folic acid supplementation on risk of cardiovascular diseases: a meta-analysis of randomized controlled trials. JAMA. 2006; 296:2720-2726.
Depeint F, Bruce W R, Shangari N, Mehta R, O'Brien P J. Mitochondrial function and toxicity: role of B vitamins on the one-carbon transfer pathways. Chem Biol Interact. 2006; 163:113-132.
Gross E R, Gross G J. Ligand triggers of classical preconditioning and postconditioning. Cardiovasc Res. 2006; 70:212-221.
Gunduz D, Kasseckert S A, Hartel F V, Aslam M, Abdallah Y, Schafer M, Piper H M, Noll T, Schafer C. Accumulation of extracellular ATP protects against acute reperfusion injury in rat heart endothelial cells. Cardiovasc Res. 2006; 71:764-773.
Gupte S A, Kaminski P M, Floyd B, Agarwal R, Ali N, Ahmad M, Edwards J, Wolin M S. Cytosolic NADPH may regulate differences in basal Nox oxidase-derived superoxide generation in bovine coronary and pulmonary arteries. Am J Physiol Heart Circ Physiol. 2005; 288:H13-H21.
Hevel J M, Marlette M A. Macrophage nitric oxide synthase: relationship between enzyme-bound tetrahydrobiopterin and synthase activity. Biochemistry. 1992; 31:7160-7165.
Hyndman M E, Verma S, Rosenfeld R J, Anderson T J, Parsons HG. Interaction of 5-methyltetrahydrofolate and tetrahydrobiopterin on endothelial function. Am J Physiol Heart Circ Physiol. 2002; 282:H2167-H2172.
JAMA: Homocysteine and risk of ischemic heart disease and stroke: a meta-analysis. JAMA. 2002; 288:2015-2022.
Jennings R B, Steenbergen C, Jr. Nucleotide metabolism and cellular damage in myocardial ischemia. Annu Rev Physiol. 1985; 47:727-749.
Jones S P, Bolli R. The ubiquitous role of nitric oxide in cardioprotection. J Mol Cell Cardiol. 2006; 40:16-23.
Jones S P, Girod W G, Palazzo A J, Granger D N, Grisham M B, Jourd'Heuil D, Huang P L, Lefer D J. Myocardial ischemia-reperfusion injury is exacerbated in absence of endothelial cell nitric oxide synthase. Am J Physiol. 1999; 276:H1567-H1573.
Jones S P, Greer J J, Kakkar A K, Ware P D, Turnage R H, Hicks M, van Haperen R, de Crom R, Kawashima S, Yokoyama M, Lefer D J. Endothelial nitric oxide synthase overexpression attenuates myocardial reperfusion injury. Am J Physiol Heart Circ Physiol. 2004; 286:H276-H282.
Klawitter P F, Murray H N, Clanton T L, Angelos M G. Reactive oxygen species generated during myocardial ischemia enable energetic recovery during reperfusion. Am J Physiol Heart Circ Physiol. 2002; 283:H1656-H1661.
Moat S J, Clarke Z L, Madhavan A K, Lewis M J, Lang D. Folic acid reverses endothelial dysfunction induced by inhibition of tetrahydrobiopterin biosynthesis. Eur J. Pharmacol. 2006; 530:250-258.
Oster K. Atherosclerosis treated with folic acid. FASEB J. 1981; 40.
Reinhardt C P, Dalhberg S, Tries M A, Marcel R, Leppo J A. Stable labeled microspheres to measure perfusion: validation of a neutron activation assay technique. Am J Physiol Heart Circ Physiol. 2001; 280:H108-H116.
Shirodaria C, Antoniades C, Lee J, Jackson C E, Robson M D, Francis J M, Moat S J, Ratnatunga C, Pillai R, Refsum H, Neubauer S, Channon K M. Global improvement of vascular function and redox state with low-dose folic acid: implications for folate therapy in patients with coronary artery disease. Circulation. 2007; 115:2262-2270.
Stroes E S, van Faassen E E, Yo M, Martasek P, Boer P, Govers R, Rabelink T J. Folic acid reverts dysfunction of endothelial nitric oxide synthase. Circ Res. 2000; 86:1129-1134.
Takimoto E, Champion H C, Li M, Ren S, Rodriguez E R, Tavazzi B, Lazzarino G, Paolocci N, Gabrielson K L, Wang Y, Kass D A. Oxidant stress from nitric oxide synthase-3 uncoupling stimulates cardiac pathologic remodeling from chronic pressure load. J Clin Invest. 2005; 115:1221-1231.
Vasquez-Vivar J, Kalyanaraman B, Martasek P, Hogg N, Masters B S, Karoui H, Tordo P, Pritchard K A, Jr. Superoxide generation by endothelial nitric oxide synthase: the influence of cofactors. Proc Natl Acad Sci USA. 1998; 95:9220-9225.
Verhaar M C, Wever R M, Kastelein J J, van Dam T, Koomans H A, Rabelink T J. 5-methyltetrahydrofolate, the active form of folic acid, restores endothelial function in familial hypercholesterolemia. Circulation. 1998; 97:237-241.
Wajima T, Shimizu S, Hiroi T, Ishii M, Kiuchi Y. Reduction of myocardial infarct size by tetrahydrobiopterin: possible involvement of mitochondrial KATP channels activation through nitric oxide production. J Cardiovasc Pharmacol. 2006; 47:243-249.
Wald D S, Wald N J, Morris J K, Law M. Folic acid, homocysteine, and cardiovascular disease: judging causality in the face of inconclusive trial evidence. BMJ. 2006; 333:1114-1117.
Wang X, Qin X, Demirtas H, Li J, Mao G, Huo Y, Sun N, Liu L, Xu X. Efficacy of folic acid supplementation in stroke prevention: a meta-analysis. Lancet. 2007; 369:1876-1882.
Yamamura K, Steenbergen C, Murphy E. Protein kinase C and preconditioning: role of the sarcoplasmic reticulum. Am J Physiol Heart Circ Physiol. 2005; 289:H2484-H2490.
Zweier J L, Talukder M A. The role of oxidants and free radicals in reperfusion injury. Cardiovasc Res. 2006; 70:181-190.

1. A method of blunting myocardial dysfunction during ischemia and/or ameliorating post-reperfusion injury in a subject, the method comprising:
administering to the subject at least 200 mg of folic acid or an equivalent dose of a folic acid derivative so as to blunt myocardial dysfunction during ischemia and/or to ameliorate post-reperfusion injury in the subject.

2. The method according to claim 1, wherein the myocardial dysfunction comprises decreased myocardial contractility.

3. The method according to claim 1, wherein the myocardial dysfunction comprises myocardial cell death.

4. The method according to claim 1, wherein the myocardial dysfunction comprises infarct-induced arrhythmias.

5. A method of ameliorating reperfusion injury in a subject, the method comprising:
administering to the subject at least 200 mg of folic acid or an equivalent dose of a folic acid derivative during active ischemia before reperfusion so as to ameliorate reperfusion injury in the subject.

6. The method according to claim 1, wherein subject is administered at least 600 mg of folic acid or an equivalent dose of a folic acid derivative.

7. The method according to claim 1, wherein the folic acid or the equivalent dose of a folic acid derivative is administered orally.

8. The method according to claim 1, wherein the administration of folic acid or the equivalent dose of a folic acid derivative is intramuscular, intravenous or transdermal.

9. A pharmaceutical composition comprising a single dose of at least 600 mg folic acid, or an equivalent dose of a folic acid derivative.

10. A method of improving or restoring decreased high energy phosphate levels in cardiovascular disorders with decreased ATP/ADP-levels, the method comprising:
administering to a subject having a cardiovascular disorder with decreased ATP/ADP levels an effective amount of folic acid to improve or restore decreased high energy phosphate levels in the subject.

11. The method according to claim 5, wherein subject is administered at least 600 mg of folic acid or an equivalent dose of a folic acid derivative.

12. The method according to claim 5, wherein the folic acid or the equivalent dose of a folic acid derivative is administered orally.

13. The method according to claim 5, wherein the administration of folic acid or the equivalent dose of a folic acid derivative is intramuscular, intravenous, or transdermal.

14. The pharmaceutical composition of claim 9, wherein the composition further comprises a pharmaceutically acceptable vector.

15. A method of improving or restoring decreased high energy phosphate levels in cardiovascular disorders with decreased ATP/ADP-levels, the method comprising:
administering to a subject having a cardiovascular disorder with decreased ATP/ADP levels an effective amount of folic acid to improve or restore decreased high energy phosphate levels in the subject; and
monitoring cardiac function in the subject.
